(19) United States
(12) Patent Application Publication
(10) Pub. No.: US 2006/0106459 A1
(43) Pub. Date: May 18, 2006

(54) Bone treatment systems and methods

(76) Inventors: Csaba Truckai, Saratoga, CA (US); John H. Shadduck, (US)

Correspondence Address: John H. Shadduck, 350 Sharon Park Drive #823, Menlo Park, CA, 94025, US

(21) Appl. No.: 11/215,730

(22) Filed: Aug. 30, 2005

Related U.S. Application Data
(60) Provisional application No. 60/605,700, filed on Aug. 30, 2004.

(51) Int. Cl. A61F 2/44

(52) U.S. Cl. 623/17.11

(57) ABSTRACT

A system for treating an abnormal vertebral body such as a compression fracture. In an exemplary embodiment, the system includes a biocompatible flow-through implant structure configured with a three-dimensional interior web that defines flow openings therein for cooperating with a two-part hardenable bone cement. The flow-through structure is capable of compacted and extended shapes and in one embodiment provides a gradient in flow openings for controlling flow parameters of a bone cement injected under high pressure into the interior thereof. The flow-through implant structure is configured for transducing cement injection forces into a selected direction for moving apart cortical endplates of a vertebra to reduce a fracture. In one embodiment, the flow-through implant structure is coupled to an Rf source for applying Rf energy to a two-part bone cement to accelerate curing of the cement to thereby allow on-demand alterations of cement viscosity. The Rf system allows for control of bone cement polymerization globally or regionally to prevent cement extravasion and to direct forces applied to a vertebra to reduce a fracture.

### CROSS-REFERENCE TO RELATED APPLICATIONS

This application claims benefit of Provisional U.S. Patent Application Ser. No. 60/605,700 filed Aug. 30, 2004 titled Vertebral Implant Constructs, Methods of Use and Methods of Fabrication. This application also is related to U.S. application Ser. No. 11/165,652 (Atty. Docket No. DFINE.001A1, filed Jun. 24, 2005 titled Bone Treatment Systems and Methods; and U.S. patent application Ser. No. 11/165,651 (Atty. Docket No. DFINE.001A2), filed Jun. 24, 2005, titled Bone Treatment Systems and Methods. The entire contents of all of the above cross-referenced applications are hereby incorporated by reference in their entirety and should be considered a part of this specification.

### BACKGROUND OF THE INVENTION

1. Field of the Invention

The present invention relates to medical devices, and more particularly, to methods and apparatus for treatment of abnormalities in bone such as osteoporotic bone, bone fractures, avascular necrosis and the like. An exemplary, deformable flow-through filament structure can be configured for implantation in a vertebra, wherein flows of bone cement into the deformable structure are controlled to prevent cement extravasion and to direct fracture-reducing forces applied to the vertebra.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the affected population will grow steadily as life expectancy increases. Osteoporosis affects the entire skeleton but most commonly causes fractures in the spine and hip. Spinal or vertebral fractures also have serious consequences, with patients suffering from loss of height, deformity and persistent pain which can significantly impair mobility and quality of life. Fracture pain usually lasts 4 to 6 weeks, with intense pain at the fracture site. Chronic pain often occurs when one level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporotic bone, the sponge-like cancellous bone has pores or voids that increase in dimension, making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. In one percutaneous vertebroplasty technique, bone cement such as PMMA (polymethylmethacrylate) is percutaneously injected into a fractured vertebral body via a trocar and cannula system. The targeted vertebrae are identified under fluoroscopy. A needle is introduced into the vertebral body under fluoroscopic control to allow direct visualization. A transpedicular (through the pedicle of the vertebrae) approach is typically bilateral but can be done unilaterally. The bilateral transpedicular approach is typically used because inadequate PMMA infill is achieved with a unilateral approach.

In a bilateral approach, approximately 1 to 4 ml of PMMA are injected on each side of the vertebra. Since the PMMA needs to be forced into cancellous bone, the technique requires high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasion are critical to the technique and the physician terminates PMMA injection when leakage is evident. The cement is injected using small syringe-like injectors to allow the physician to manually control the injection pressures.

Kyphoplasty is a modification of percutaneous vertebroplasty. Kyphoplasty involves a preliminary step that comprises the percutaneous placement of an inflatable balloon tamp in the vertebral body. Inflation of the balloon creates a cavity in the bone prior to cement injection. Further, the proponents of percutaneous kyphoplasty have suggested that high pressure balloon-tamp inflation can at least partially restore vertebral body height. In kyphoplasty, it has been proposed that PMMA can be injected at lower pressures into the collapsed vertebra since a cavity exists to receive the cement—which is not the case in conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles. Leakage of PMMA during vertebroplasty can result in very serious complications including compression of adjacent structures that necessitate emergency decompressive surgery.

Leakage or extravasion of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (5/2004) pp. 478-82, (http://www.jkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2): 175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al., “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection are also cause for concern. See Kirby, B., et al., “Acute bronchospasm due to exposure to polymethylmethacrylate vapors during percutaneous vertebroplasty”, Am. J. Roentgenol. 2003; 180:543-544.

Another disadvantage of PMMA is its inability to undergo remodeling—and the inability to use the PMMA to deliver osteoinductive agents, growth factors, chemotherapeutic agents and the like. Yet another disadvantage of PMMA is the need to add radiopaque agents which lower its viscosity with unclear consequences on its long-term endurance.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon also applies compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which first crushes and compacts cancellous bone. Expansion of the balloon under high pressures close to cortical bone can fracture the cortical bone, or cause regional damage to the cortical bone that can result in cortical bone necrosis. Such cortical bone damage is highly undesirable and results in weakened cortical endplates.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide systems and methods for use in treatment of vertebral compression fractures that provide a greater degree of control over introduction of bone support material, and that provide better outcomes. Embodiments of the present invention meet one or more of the above needs, or other needs, and provide several other advantages in a novel and non-obvious manner.

### SUMMARY OF THE INVENTION

In general, the invention comprises a biocompatible implant structure configured with a three-dimensional interior web that defines flow openings therein for cooperating with a two-part hardenable bone cement. The structure is capable of compacted and extended shapes and in one embodiment provides a gradient in flow openings for controlling flow parameters of a bone cement injected under high pressure into the interior of the web structure. The flow-through implant structure is configured for transducing the injection forces into a selected direction for moving apart cortical endplates of a vertebra to reduce a fracture.

In one embodiment, the implantable flow-through structure reduces or eliminates the possibility of PMMA extravasion from a targeted treatment site. In another embodiment, the system can be used for minimally invasive prophylactic treatment of osteoporotic vertebrae that are susceptible to compression fractures. In another embodiment, the system allows for control of thermal diffusion from an exothermic bone cement to control thermal damage to bone.

In another embodiment, the flow-through implant structure can be coupled to an Rf source to function as at least one electrode in a mono-polar or bi-polar arrangement. The system can apply Rf energy to a two-part bone cement to accelerate curing of the cement for positive control of cement flow parameters. The Rf system allows for control of bone cement polymerization to globally or regionally impart to a cement volume a desired viscosity to prevent cement extravasion.

In another embodiment, the system provides a radiopaque implant structure that can reduce the volume of radiopaque agents needed in a bone cement formulation which can result in a higher strength bone cement.

These and other objects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a sectional perspective view of a hypothetical, three-dimensional deformable flow-through structure in a first compacted configuration, the structure capable of a second extended or expanded configuration.

FIG. 2 is a sectional perspective view of the deformable flow-through structure of FIG. 1 in an extended configuration, the structure then defining a gradient in flow openings therein.

FIG. 3 is a cut-away side view of a vertebra with a compression fracture showing an introducer in a transpedicular approach with a flow-through implant structure similar to FIGS. 1 and 2 in a pre-deployed position within an introducer.

FIG. 4 is a view of the vertebra of FIG. 3 with the compression fracture reduced after injection of a bone cement into the flow-through implant structure wherein the system applies retraction forces to increase vertebral height.

FIG. 5 is an enlarged sectional view of the implant structure and vertebra similar to FIG. 3 wherein the implant structure is initially deployed into bone from an introducer.

FIG. 6 is an enlarged sectional view of the implant structure of FIG. 5 following injection of an in-situ polymerizable cement into the interior of the implant.

FIG. 7 is a sectional view of an alternative 3D construct of a filament material for providing non-linear material properties to the polymerized implant, the filaments defining a gradient in opening cross sections therein.

FIG. 8 is an exploded view of another flow-through structure for cooperating with a bone cement, the structure carried by the working end of an introducer and coupled to a Rf source for delivering energy to bone cement flows within the structure.

### DETAILED DESCRIPTION OF THE INVENTION

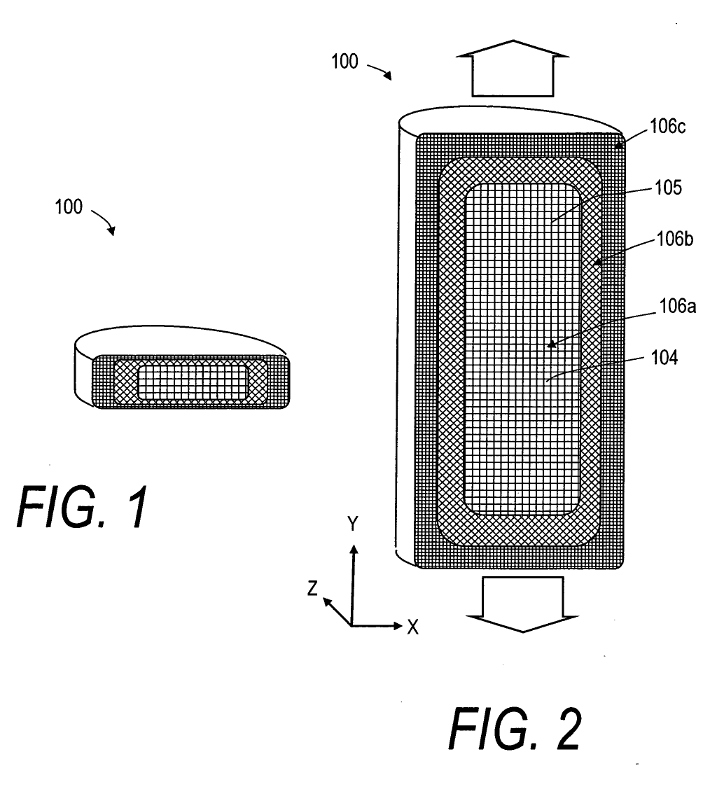

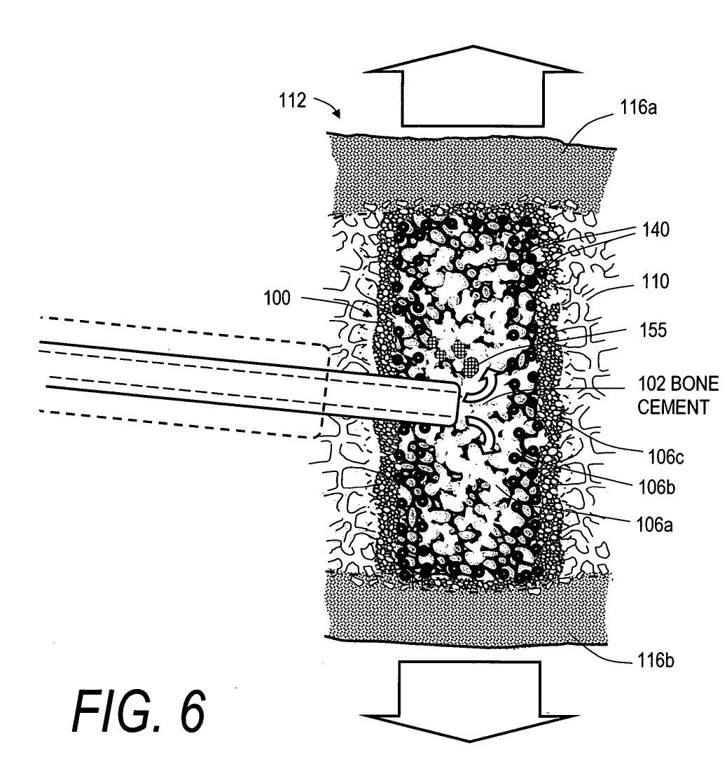

FIGS. 1 and 2 depict schematic sectional views of an exemplary, deformable flow-through implant body or structure 100 that is configured for treating a fracture in a vertebral body. In FIG. 1, it can be seen that the deformable structure 100 is capable of a collapsed or compacted shape to allow for its introduction into a vertebra through a small diameter sleeve. FIG. 2 illustrates that the deformable structure 100 is capable of extension in a controlled direction relative to x, y and z-axes of the body following the flow of fill material 102 (see FIG. 6) into implant body 100. The fill material 102 can be an in-situ hardenable bone cement, such as a PMMA bone cement that is injected in a common form consisting of (i) a liquid MMA monomer component and (ii) a non-liquid pre-polymerized PMMA bead component. The flow-through structure 100 of FIG. 2 comprises an open web of elements 104 that define flow openings 105 therein. The elements 104 can be filaments or polymer ligaments of a foam material as will be further described below. In use, the combination of deformable structure 100 and the flow of bone cement 102 into the structure 100 can function as a jack to engage and move apart cortical endplates to reduce a vertebral fracture.

A two-part bone cement 102 that can be used comprises a volume of a liquid component for chemically interacting with the surface area of PMMA beads particles. The liquid component precursor typically includes an MMA monomer and DMPT. In one embodiment, the pre-polymerized PMMA beads or particles comprise from 65 to 72 percent of the non-liquid component, BPO comprises 0.5 to 3.0 percent of the non-liquid component and a radiopaque material such as BaSO₄ comprises 25 to 30 percent or non-liquid component. In this embodiment of cement, the liquid component comprises from about 97 to 99.5 percent MMA with a large part of the remainder being DMPT (dimethyl-p-toluidine) and hydroquinone as is known in the art.

FIG. 2 illustrates that implant structure 100 has a gradient in material properties such as the dimensions of flow openings 105 across a transverse axis (x-axis) and longitudinal axis (z-axis) of the body. By the term gradient, it is meant that implant has at least one interior region that has properties that differ from a surface region—and the gradient may be a continuous change in the property or several regions of progressively varying properties. In FIG. 2, the deformable structure 100 has a core interior region 106a, an intermediate region 106b and a surface region 106c. One gradient material property of interest is the dimension of flow openings 105 in structure 100 in advance of cement flows therein. Other gradient material properties are also of interest, for example, following the hardening of bone cement 102 within and about the extended deformable structure 100, the system can provide a gradient in Young's modulus or strength of the implant-particularly in the y-axis direction for supporting physiologic loads. The variation in modulus can be provided by a variation in properties of the (non-liquid) pre-polymerized bead component of a PMMA bone cement, wherein the varied fill materials are introduced in different aliquots of cement. Also, a gradient can be provided by varying the porosity of pre-polymerized beads or metallic beads that are introduced in different aliquots of a bone cement. The varied porosity can be optimized for bone ingrowth in the surface of the cured, implanted material. Another material property that can have a gradient relates to the level of thermal insulation provided by the non-liquid bead component of an exothermic bone cement. The bead component can include highly insulative glass or ceramic microspheres for confining heat more within the interior region of the implant structure to provide less thermal diffusion from the surface of the curing implant material.

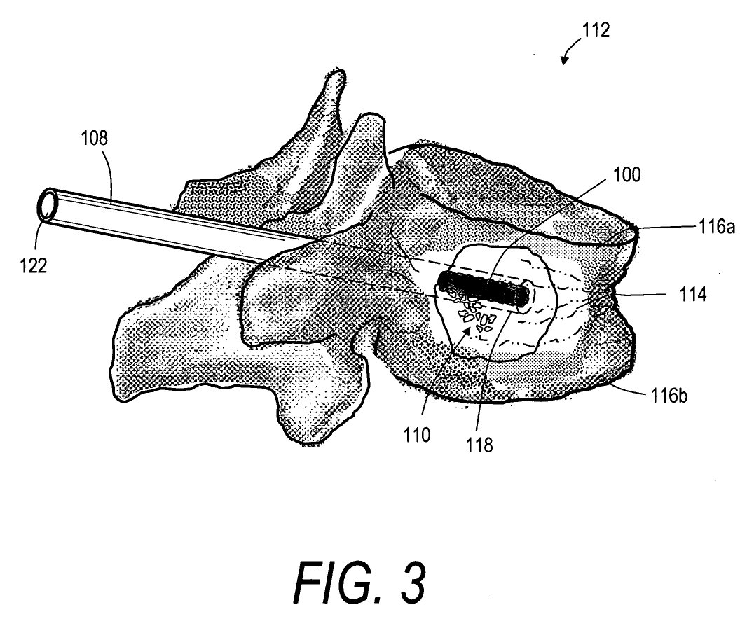

In a method of use, FIG. 3 illustrates the introduction of deformable structure 100 in a first compacted shape through an introducer 108 into cancellous bone 110 of a vertebra 112. The vertebra 112 has a compression fracture 114 that has caused collapse of vertebral height in an anterior portion thereof. The vertebral endplates are indicated at 116a and 116b. The cancellous bone 110 at the interior of the vertebra is osteoporotic and has been crushed to some extent by the fracture. In FIG. 3, it can be seen that an introducer sleeve 108 has been introduced in a transpedicular approach with distal working end 118 in an anterior region of cancellous bone 110. In this view, the introducer sleeve 108 carries the compacted structure 100 in its bore 122 that can be compared to the structure of FIG. 1 if it were further compacted.

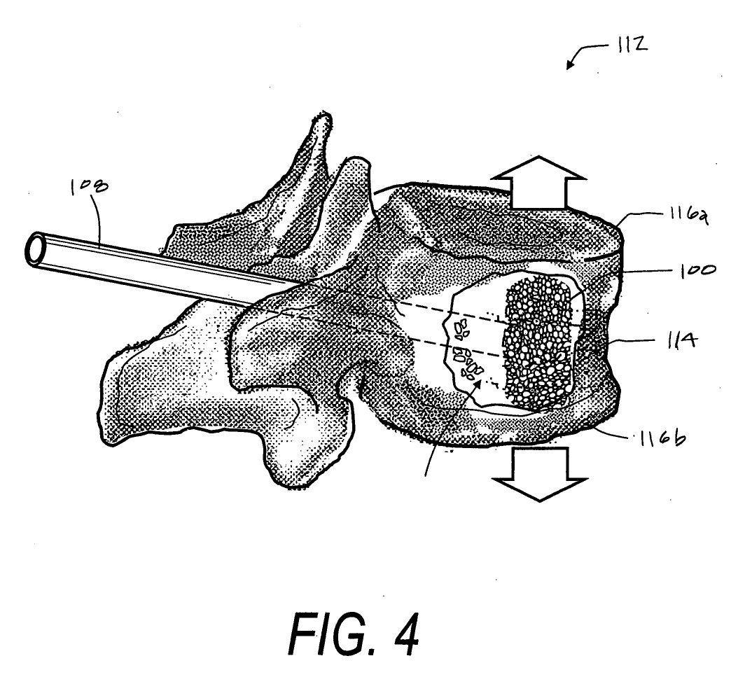

In a subsequent step of the method, FIG. 4 illustrates the high-pressure injection of cement 102 into the interior of the deformable structure 100 which extends the structure toward a second extended shape. In this view, the deformable structure 100 can be compared to the hypothetical structure of FIG. 2. Next, it will be described how the structure 100 of FIG. 3 cooperates with flows of fill material or cement 102 to treat a vertebral fracture. In this disclosure, the deformable structure can be any form of flow-through body such as an open-cell polymer monolith, a knit structure, a woven structure, a braided structure or any combination thereof that has webs, ligaments, struts, elements 104 or the like that extend in three dimensions throughout the interior volume of the structure 100 to thereby define flow openings 105 between the adjacent webs, ligaments, struts or elements 104. The webs 104 and flow openings 105 can be provided in a gradient in dimensions and can effectively constrain the structure in a predetermined extended shape. The novel three dimensional flow-through structure 100 is thus distinguished from shell-like structures without such three-dimensional web elements extending throughout the interior volume of the structure.

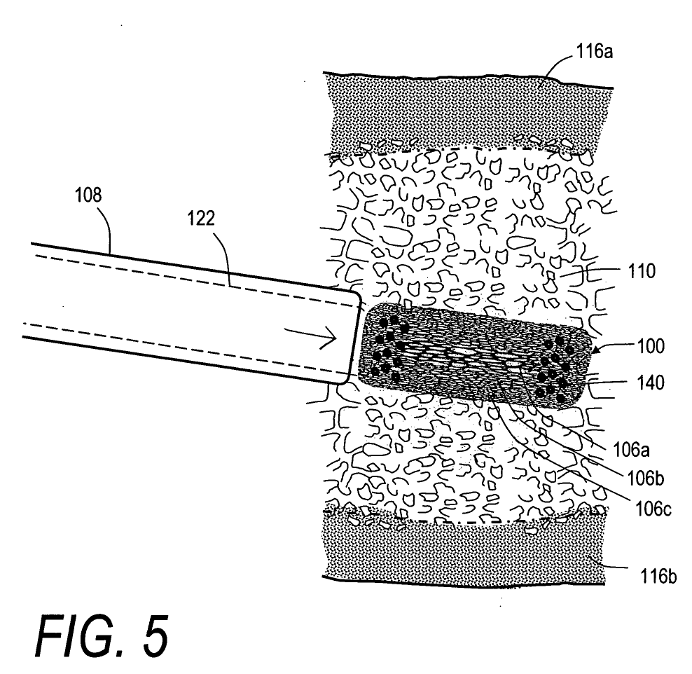

Now turning to FIG. 5, an enlarged view is shown of the exemplary deformable structure 100 being deployed from introducer 108. The structure 100 defines a web of elements 104 that define flow openings 105 having a gradient in open dimensions from the interior to the surface thereof with predetermined larger openings in interior region 106a and with predetermined smaller openings in surface region 106c. In one embodiment as in FIG. 5, the structure 100 is fabricated of an open-cell polymer. In another embodiment, the structure 100 can be a web of polymer, metal or carbon fiber filaments. FIGS. 5 and 6 depict an embodiment with a further filament structure 140 (metal or polymer) therein that is helical or woven and serves to direct forces caused by inflows of a high viscosity flowable cement 102. The mean dimensions of flow openings 105 in outer region 106c are selected to allow a limited flow therethrough of a flowable bone cement to interdigitate with bone when the cement has a selected viscosity and is introduced under a selected pressure. The gradient in open dimensions in flow openings 105 of the webs are further selected to filter and trap selected solid bead materials 155 within a flowable cement injected into the interior region of the structure 100. By this means, smaller solid bead elements 155 will aggregate toward the surface of structure 100 and larger bead elements will aggregate toward the interior of the structure. It can be understood that beads 155 aggregating in the surface regions of structure 100 will prevent extravasion of the cement after cement has filled the structure. Of particular interest, the reinforcing filament structure 140 therein will cause inflow pressure of the cement to direct forces in the direction of the arrows in FIG. 6 to apply jacking forces to the interior of the vertebra to reduce the fracture. FIG. 6 illustrates the implant structure 100 after it extends to an increased height to engaging endplates 116a and 116b of the vertebra 112.

In another embodiment, the form of structure 100 can provide the smaller beads that aggregate at the periphery with an open porous network that carries at least in part a material configured for timed release such as a pharmacological or bioactive agent (e.g., any form of BMP, an antibiotic, an agent that promotes angiogenesis, etc.).

In FIG. 6, a single structure 100 is shown in an extended, predetermined elongated shape after the introduction of a flowable bone cement 102 into the interior of the structure. In use, the physician can introduce a plurality of such structures 100, for example one or more on each side of a vertebra in a bilateral transpedicular approach. The scope of the invention includes introducing a plurality of such structures 100 in a unilateral transpedicular approach, or one or more deformable structures can be uses in treatments of other bones.

The method of the invention further includes controlling thermal effects of an exothermic in-situ polymerizable cement such as a PMMA cement. In one embodiment, a polymeric foam structure 100 is provided that carries insulative microspheres in the webs 104 of the open cells which can substantially reduce heat transfer from an exothermic cement to adjacent bone. In another embodiment, the level of heat transfer is controlled by providing a volume of insulative microspheres of glass, ceramic or a polymer that is injected as a portion of the non-liquid component of the two-part PMMA cement described above, or in a first aliquot of the introduced cement. Such insulative microspheres will then aggregate in the periphery of the structure 100 to limit thermal heat transfer outwardly to bone. Insulated microspheres are available from Potters Industries Inc., P.O. Box 840, Valley Forge, Pa. 19482, for example, microspheres marketed under the names of Spheriglass®, Sphericel® and Q-Cel®.

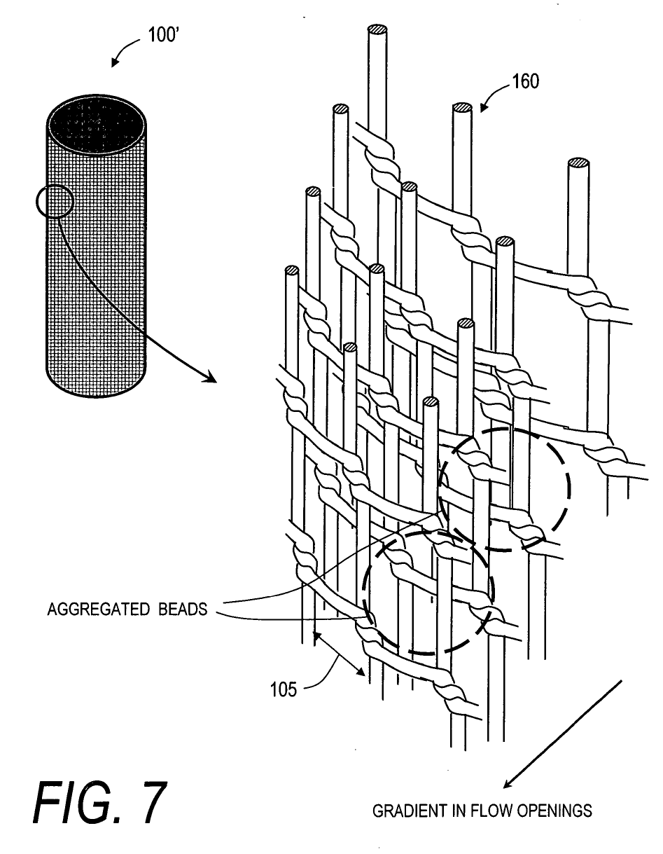

FIG. 7 illustrates another embodiment wherein the deformable structure 100′ is of filament 160 that can be knit, woven or braided in a suitable manner to provide a filament structure that is equivalent to an open cell polymer that extends monolithically in x, y and z-axes through the interior of the construct body to thereby cooperate with a two-part bone cement a described above. For example, a PMMA cement will comprise a liquid monomer with PMMA beads that have a diameter ranging between about 100 and 2500 microns, and more preferably between about 250 and 1000 microns. The deformable structure 100′ can be configured so that a first aliquot of cement carrying smaller beads will flow through the structure in a selected amount to interdigitate with cancellous bone and then a subsequent aliquot with larger beads will tend to aggregate in surface 106c of structure 100′. Thereafter, additional volumes or aliquots of cement are introduced which can carry larger diameter beads. It can be understood that high injection pressures will result in directing the extension forces in a manner to reduce the vertebral fracture as described above.

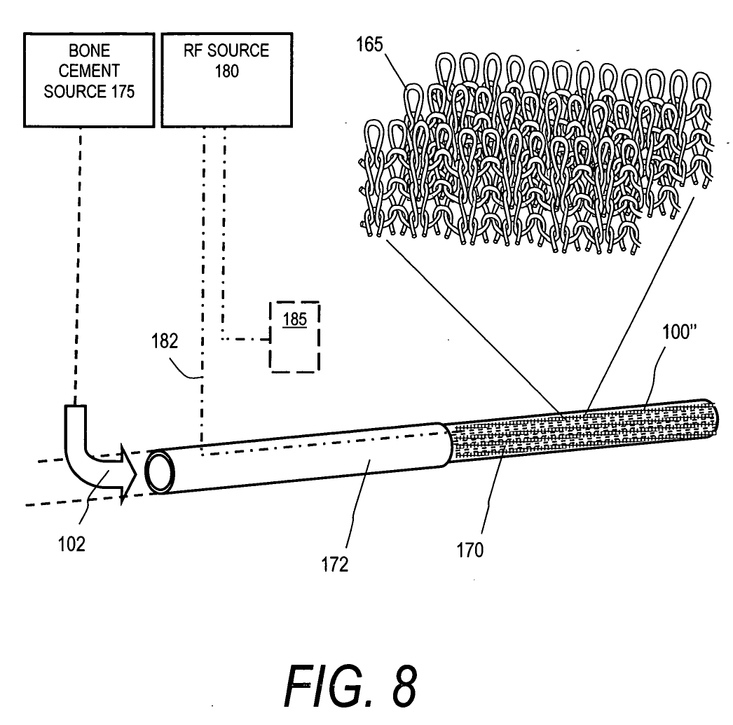

FIG. 8 illustrates another embodiment wherein the deformable structure 100″ is fabricated of knit conductive filaments 165 that provide a flow-through filament structure as described previously. The structure 100′ is coupled to, or deployable from, a distal working end 170 of an introducer 172. In this embodiment, a pressurizable source 175 of bone cement 102 is provided together with a radiofrequency (Rf) source 180 coupled by at least one electrical lead 182 to the conductive filaments 165 of deformable structure 100″. It has been found that controlled Rf energy delivery to a flow of an exothermic bone cement can practically instantly alter the viscosity of the cement to control flow properties of the cement. In order to deliver Rf energy to a cement, the cement needs to carry a conductive filler or a filament flow-through structure can be provided. Co-pending U.S. patent application Ser. No. 11/165,652 (Atty. Docket No. DFINE.001A1, filed Jun. 24, 2005 titled Bone Treatment Systems and Methods, and U.S. patent application Ser. No. 11/165,651 (Atty. Docket No. DFINE.001A2), filed Jun. 24, 2005, titled Bone Treatment Systems and Methods, describe apparatus and methods of using Rf energy delivery to bone cement for controlling flow properties. The specifications of these patents can be referenced for Rf operational parameters that are applicable to filament structure 100″ depicted in FIG. 8. In one mode of operation, the Rf energy can be delivered to filament structure 100″ and cement flows therein in a mono-polar manner in cooperation with a return electrode 185 (FIG. 8) as in known in the art. In another mode of operation, Rf energy can be delivered to filament structure 100″ and cement flows therein in a bi-polar manner. In such a bi-polar method, the filament structure 100″ has first and second opposing polarity electrical leads extending from Rf source 180 to spaced apart first and second conductive filament regions that thus exhibit opposing polarities. The first and second conductive filament regions are separated by non-conductive knit filaments regions. Computer controlled technical knitting machines can be used to fabricate the filament structure 100″ of FIG. 8. The first and second opposing polarity filament regions can be separated in radial angles about the filament structure 100″, or can be separated concentrically relative to each other in the filament structure 100″, or can be separated axially or helically in the filament structure 100″. In any such arrangement, either the surface of a volume of inflowing bone cement can be cured on demand, or any quadrant or section of the surface can be cured on demand depending on the orientation of the first and second conductive filament regions. The opposing polarity conductive filament regions each can comprise multiple sub-regions for providing bi-polar Rf delivery to selected portions of a bone cement flow in the filament structure 100″. In the embodiment of FIG. 8, the filament structure 100″ can be deployable from the introducer or the filament structure 100″ can be a part of a working end that is releasable and implantable in the vertebra. In another embodiment, the filament structure 100″ can extend outwardly from the side of the introducer.

In another method of the invention, the implant structure is of a radiopaque material or is a polymer doped with a radiopaque composition to allow for imaging of the structure as in known in the art.

The above description of the invention intended to be illustrative and not exhaustive. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

1-33. (canceled)

34. A bone treatment system comprising:
a deformable structure configured for introduction into a bone, the structure including an interior web of elements that define flow openings therebetween, the flow openings defining a gradient between larger interior flow openings and smaller exterior flow openings;
a bone fill material for introduction into the interior of the deformable structure.

35. The bone treatment system of claim 34 wherein the deformable structure is at least one of a knit structure, woven structure, braided structure and foam structure.

36. The bone treatment system of claim 34 wherein the interior web is fabricated of at least one of metal filaments, polymer filaments, and polymer foam.

37. The bone treatment system of claim 34 wherein the deformable structure is fabricated of an electrically conductive material.

38. The bone treatment system of claim 37 further comprising an electrical energy source coupled to the deformable structure.

39. The bone treatment system of claim 34 wherein the deformable structure is capable of deformation between a compacted condition and an extended condition.

40. The bone treatment system of claim 34 wherein the bone fill material includes bone cement having a liquid component and a non-liquid component.

41. The bone treatment system of claim 40 wherein the non-liquid component includes substantially spherical beads.

42. The bone treatment system of claim 41 wherein the spherical beads have at least one selected diameter for cooperating with the flow openings.

43. The bone treatment system of claim 41 wherein the spherical beads are pre-polymerized PMMA.

44. A method of treating an abnormal vertebra comprising the steps of:
introducing a deformable structure into the interior of a vertebra, the implant structure including an interior web of elements that define flow openings therebetween, the flow openings defining a gradient between larger interior flow openings and smaller exterior flow openings; and
flowing a fill material into the interior of the deformable structure wherein the fill material includes a liquid component and non-liquid component and the flow openings at least partly control flow parameters of the fill material.

45. The method of treating an abnormal vertebra of claim 44 wherein flowing the fill material reduces a fracture.

46. The method of treating an abnormal vertebra of claim 44 wherein flowing the fill material moves at least one of cancellous bone and cortical bone.

47. The method of treating an abnormal vertebra of claim 44 wherein the non-liquid component at least partly aggregates in selected flow openings.

48. The method of treating an abnormal vertebra of claim 44 wherein flowing the fill material deforms the deformable structure from a compacted shape to a selected extended shape.

49. The method of treating an abnormal vertebra of claim 20 wherein the extended shape has a greater vertical dimension and a lesser horizontal dimension.

50. A bone treatment system comprising:
a deformable structure including a web of conductive elements that define flow openings therebetween;
a radiofrequency (Rf) energy source operatively coupled to the conductive elements; and
a fill material for introduction into the interior of the deformable structure.

51. The bone treatment system of claim 50 wherein the conductive elements are coupled to a single pole of the Rf source for operating in a mono-polar manner in cooperation with a remote return electrode.

52. The bone treatment system of claim 50 wherein the conductive elements have first and second opposing polarity portions coupled to opposing poles of the Rf source for operating in a bi-polar manner.

53. The bone treatment system of claim 50 wherein the deformable structure is at least one of a knit structure, woven structure and braided structure.
